(19) United States
(12) Patent Application Publication
(10) Pub. No.: US 2021/0298762 A1
(43) Pub. Date: Sep. 30, 2021

(54) REVERSIBLE LACRIMAL CANALICULAR OCCLUSION DEVICE

(71) Applicant: THE JOHNS HOPKINS UNIVERSITY, Baltimore, MD (US)

(72) Inventors: Albert S Jun, Towson, MD (US); Bradley P. Barnett, Apex, NC (US)

(21) Appl. No.: 17/265,650

(22) PCT Filed: Jul. 15, 2019
(86) PCT No.: PCT/US2019/041789

§ 371 Date: Feb. 3, 2021

Related U.S. Application Data
(60) Provisional application No. 62/714,136, filed on Aug. 3, 2018.

(51) Int. Cl. A61B 17/12; A61F 9/00

(52) U.S. Cl. CPC A61B 17/12099; A61B 2017/00867; A61F 9/0017; A61B 17/12031

(57) ABSTRACT

Canalicular occlusion devices are described comprising an occlusive element and a memory frame comprising a memory material. The occlusive element is configured to substantially prevent fluid from flowing through portions of the canaliculus. Also disclosed are methods of occluding a canalicular system with the devices of the present invention.

### REFERENCE TO RELATED APPLICATIONS

This application claims the benefit of U.S. Provisional Patent application 62/714,136 filed Aug. 3, 2018, which is hereby incorporated by reference for all purposes as if fully set forth herein.

### BACKGROUND OF THE INVENTION

The prevalence of dry eye disease is estimated to be between 7.4% to 33.7% depending on diagnostic criteria and patient population. Punctal occlusion is an effective treatment for dry eye disease. Current permanent punctal occlusion devices have a largely static shape thereby requiring multiple different size devices to accommodate the variation in punctal and canalicular anatomy. In addition, conventional permanent punctal plugs have a large fixation plate that extends beyond the punctal os and thus can cause discomfort.

FIG. 1 shows an example of a conventional plug known as a SnugPlug™ that expands once deployed from an insertion device to fill a punctum. This plug is bulky and made out of bulky elastomers to avoid puncturing by an insertion device. It cannot be delivered deep in the canalicular system because of its size and shape and is made specifically for delivery into a subject's punctal os. As shown in FIG. 1, a SnugPlug™ insertion device 150 comprises an insertion cylinder 160 in which a SnugPlug™ 170 is placed over the insertion cylinder 160. Insertion of a SnugPlug™ 170 into a punctal os occurs by the proximal end 180 of an insertion cylinder 160 pressing against the inner wall of SnugPlug™ 170 and stretching the SnugPlug™ 170 to minimize its diameter (without breaking the plug) as it is placed in a subject's punctal os. Retracting the insertion cylinder 160 minimizes the tension on the SnugPlug™ 170 to allow the SnugPlug™ 170 to expand and increase its diameter size to fit tightly in the punctum. A subject with a SnugPlug™ 170 in his or her punctum have reported both discomfort and loss of plugs loss during plug usage. New plug designs are needed to enhance patient comfort and alleviate morbidity from dry eye disease.

### SUMMARY OF THE INVENTION

Canalicular occlusion devices of the present invention include many benefits such as: 1) universal fit so that one device fits all patients including upper and lower punctum alike; 2) easily inserted and easily retrievable from the punctum; 3) superior comfort and low profile since the device rests beneath or within the punctum, 4) minimal tissue injury during the placement and the recovery of the device, 5) durable occlusion of tears in the eye of a subject comprising a canalicular occlusion device, 6) low rate of migration of the canalicular occlusion device in the punctum of a subject, 7) the canalicular occlusion device is inert to topical ocular medications and has low allergic potential, 8) the canalicular occlusion device is bacteria resistant, and 9) the canalicular occlusion device is easy to recover if located deep in a canalicular system of a patient.

One embodiment of the present invention is a canalicular occlusion device comprising and occlusive element and a memory frame comprising a memory material. The occlusive element is configured to substantially prevent or stop fluid from flowing through portions of the canaliculus. The occlusive element is attached to the memory frame and may completely or partially cover it. The memory frame may have a length in a range equal to, or shorter than, the distance from a subject's ocular punctum to the end of the subject's canalicular space. Consequently, occlusion may be made to occur in any region of a subject's canaliculus. A memory frame and/or occlusive element may have any suitable shape to fit comfortably in a canaliculus. For example, a memory frame and/or occlusive element may be linear, football, oblong, or rectangular shaped, as examples. A memory frame may comprise a memory material selected from the group consisting of polynorbomene, polycaprolactone, polyenes, nylons, polycyclooctene (PCO), blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, and combinations thereof. Alternatively, a memory frame may comprise a memory material metal selected from the group consisting of stainless steel, cobolt, nickel, chromium, molybdenumtitanium, nitinol, tantalum, platinum-iridium alloy, gold, magnesium, and combinations thereof.

An occlusion element of the present invention may be made of memory material selected from the group consisting of polytetrafluoroethylene, polyethylene terephthalate, polyethylene, silicone, acrylate polymer, urethane polymer, rayon, rubber, latex, polyurethane, thermoplastic polyurethane, polyvinylchloride and combinations of the above. An occlusive element may also comprise a waterproof coating. A canalicular occlusion device of the present invention may comprise a fixation element wherein a linear frame is located between the memory frame and the fixation element. The memory frame, the fixation element, the linear frame, and the occlusive element may be made of the same material or different materials. In some embodiments, the memory frame is made of nitinol and the fixation element is a polymer. A memory frame may have a rigidity in the range of 1 kPa to 10 kPa and has a length in the range of 0.1 mm and 10 mm. A canalicular occlusion device may have a diameter in the range of 0.1 mm to 10 mm.

Another embodiment of the present invention is a method of occluding a canalicular system, the method comprising: delivering a canalicular occlusion device of the present invention configured in a constrained form associated with a delivery device to a target location within the canalicular system, wherein when the canalicular occlusion device is deployed it thereby assumes an expanded form making contact to the walls of the canalicular system. When the canalicular occlusion device is in a constrained form it is unable to prevent fluid from flowing through the canaliculus, or allows fluid to flow around the canalicular occlusion device and through the canaliculus. When the canalicular occlusion device is in an expanded form, it prevents fluid from flowing through the canaliculus and around a canalicular occlusion device.

### Definitions

The term “distal” refers anatomically to the region deep within a canalicular system. When referring to a device of the present invention, the term “distal” refers to the portion of the device (such as occlusive element in the shape of a cap 302 shown in FIG. 2, as an example) that is intended to be inserted and deployed deep within the canalicular system.

The term “proximal” refers anatomically from within the canalicular system towards the puncta and external to the patient. When referring to a device of the present invention it represents the portion of the delivery device near a fixation element (such fixation element in the shape of arms 308 in FIG. 2 or a fixation element in the shape of a loop 408 in FIG. 3.

The term “reduces” refers to a negative alteration of at least 10%, 25%, 50%, 75%, or 100%.

The term “reference” refers to a standard or control conditions such as a sample (punctum) or a subject that is a free, or substantially free, of a device of the present invention.

The term “subject” is intended to refer to any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith such as dry eye disease. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition such as dry eye, for example.

### BRIEF DESCRIPTION OF THE DRAWINGS

FIG. 1 illustrates an example of a conventional stretch plug design.

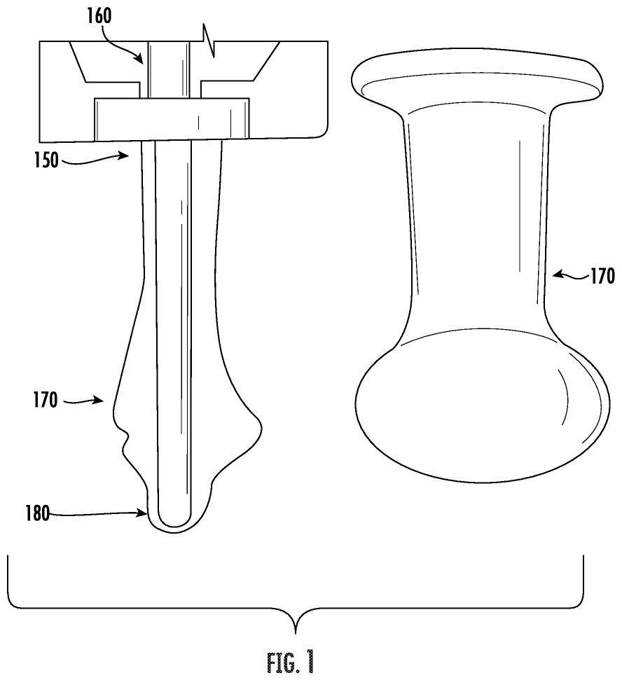

FIG. 2 illustrate an example of a canalicular occlusion device design of the present invention.

FIG. 3 illustrates an example of a canalicular occlusion device within the canaliculus of a subject.

FIG. 4A-4B illustrates alternate designs of the canalicular occlusion device of the present invention.

FIG. 5A-5D illustrates a prototype helix device shown with a penny for relative size deployed into an acrylic canalicular model and recovered with flexible shaft microforceps.

FIG. 6A-6C illustrates a nitinol capture loop opening from within catheter.

FIG. 7A-7B illustrates an occlusion testing system including an acrylic model 940 with a cylinder space 946. (A) The acrylic model 940 is attached to a syringe pump elastomeric tubing 942 and pressure gauge 944. Pressure is increased in the cylinder space 946 of the acrylic model before and after the cylinder space 946 is plugged with a second prototype occlusion device 948 of the present invention. (B) The graph demonstrates two results obtained from placing a second prototype occlusion device 948 in the cylinder space 946. First, the pressure at which the second prototype occlusion device 948 fails to occlude in the cylinder space 946 (the cylinder space is a model for a canalicular space) was determined as represented as an asymptote of graph. Second when the prototype occlusion device 948 was removed, complete reversibility of occlusion was observed as the latter pressure falls to zero after removal of prototype occlusion device 948.

### DETAILED DESCRIPTION OF THE INVENTION

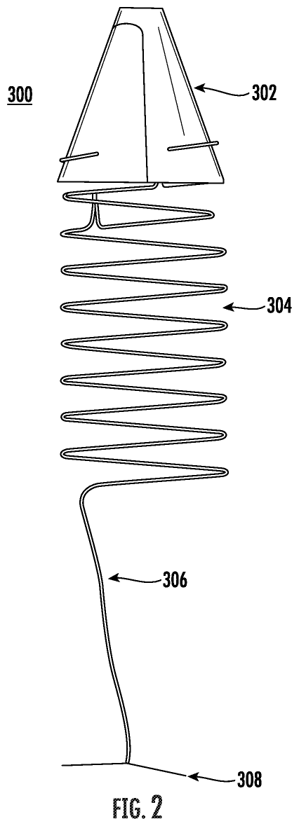

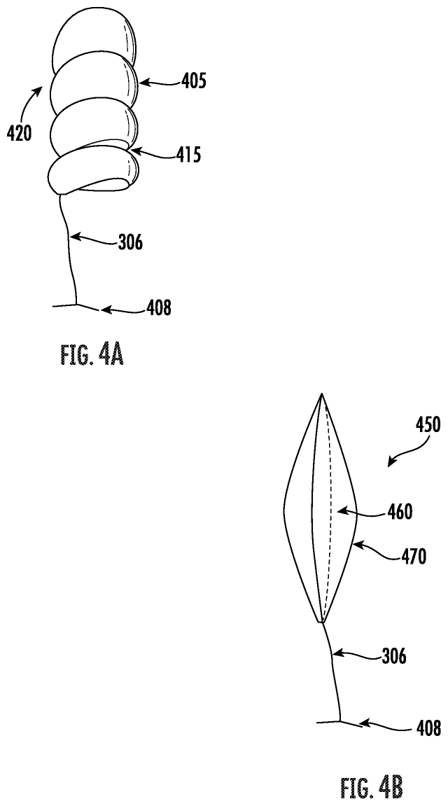

The inventors discovered a self-expanding lacrimal canalicular occlusion device designed to be a one-size fits all. The occlusion device may be used for occlusion of the canalicular anatomy as a treatment for eye diseases including dry eyes. Examples of canalicular occlusion devices of the present invention are provided in FIGS. 2 and 4. FIG. 2 shows a helical shaped occlusion device 300 wherein the occlusive element 302 and the memory frame 304 are in the shape of a helix. The linear frame 306 is linear, and the fixation element is in the shape of arms 308. FIGS. 4A and 4B illustrate additional examples of canalicular occlusion device designs. In FIG. 4A, an occlusive device with a spiral occlusive membrane 405 is illustrated. An occlusive element having a spiral shape 415 covers a memory frame 420 that extends into the occlusive element. The occlusive device includes a linear frame 306 and fixation elements in the shape of arms 408. In FIG. 4B, an occlusion device in the shape of a football 450 is illustrated. The occlusive element has a football shape when in an expanded form 450 (expanded form is shown). The occlusion element 470 covers or surrounds a memory frame 460. In some embodiments, an occlusion element includes a linear frame 306 and a fixation element in the shape of arms 408.

All, or some of, the elements of a canalicular occlusion device of the present invention may comprise memory material metal, such as a memory frame. Suitable memory material metals (a memory material) include stainless steel, cobolt, nickel, chromium, molybdenumtitanium, Nitinol, tantalum, platinum-iridium alloy, gold, magnesium, MP35N, MP20N, or combinations “MP35N” consists of 35% cobalt, 35% nickel, 20% chromium, and 10% molybdenum. “MP20N” consists of 50% cobalt, 20% nickel, 20% chromium, and 10% molybdenum. All, or some of, the elements of a canalicular occlusion device of the present invention may comprise a memory polymer. Memory polymers suitable for use in the present invention includes polynorbomene, polycaprolactone, polyenes, nylons, polycyclooctene (PCO), blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, and blends thereof. Memory metals and or memory polymers used in the present invention may be covered in a biocompatible membrane. Suitable biocompatible polymers include poly(ethylene terephthalate), polylactide, polyglycolide and copolymers thereof fluorinated polymers, such as PTFE, expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments.

In addition, materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. Thus, any polymer that may be formed into a porous sheet can be used to make a graft material, provided the final porous material is biocompatible. Polymers that can be formed into a porous sheet include polyolefins, polyacrylonitrile, nylons, polyaramids and polysulfones, in addition to polyesters, fluorinated polymers, polysiloxanes and polyurethanes as listed above. Preferably, the porous sheet is made of one or more polymers that do not require treatment or modification to be biocompatible. The graft material may include a biocompatible polyurethane. Examples of biocompatible polyurethanes include THORALON″ (Thoratec, Pleasanton, Calif.), BIOSPAN″, BIONATE^{β}, ELASTHANE™, PURSIL™ and CARBOSIL™ (Polymer Technology Group, Berkeley, Calif.). A biocompatible polymer used in the present invention may have a Young's modulus in the range of 0.01 kPa to 1 kPA.

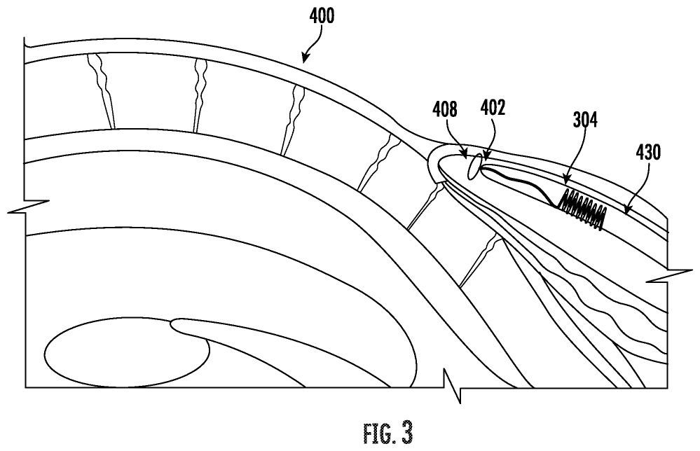

The canalicular occlusion devices of the present invention are collapsible and stretchable to be comfortably inserted into canalicular anatomy using a low-profile delivery device. The canalicular occlusion device is stretched by a low profile delivery device, so that it becomes narrow, almost linear prior to delivery of the canalicular occlusion device to a subject (i.e. constrained form). When the delivery device retracts, the helix is free to expand to its native shape (i.e. expanded form) based upon its predetermined shape as shown in FIG. 3. FIG. 3 illustrates a picture of an eye including a cornea 400 a punctum 402 and a canaliculus 430. An occlusion device of the present invention, in an expanded form, is located in the canaliculus with a fixation element in the shape of a loop 408 located outside the canaliculus. The memory frame in the shape of a helix 304 is located in the canaliculus. Ideally, the memory frame in the shape of a helix 304 expands to durably occlude the canaliculus without creating undue pressure on the canalicular walls so as to avoid canalicular trauma and dilatation. As the canaliculi measure 8-10 mm in length and 0.5-1.0 mm in diameter the ideal canalicular occlusion device design would fall within or just beyond these bounds, for example between 5-12 mm in length and 0.3 and 2.0 mm in width. Longer designs could be employed to occlude the common canaliculus to provide occlusion of the upper and lower punctum with a single device.

The geometry of a human canalicular system will dictate the optimal design of the occlusion element and/or memory frame. In an ideal embodiment, a memory frame and/or occlusion element would enlarge to occupy all potential canalicular anatomy. Specifically, it would provide a sufficient radial force in the largest canalicular segment to provide durable occlusion and stable positioning while not providing too great of outward radial force to damage the smallest canalicular segment. A canalicular occlusion device of the present invention may be in the range of size from 0.1 mm to 1 mm. Unrestrained the diameter of coil would likely be 1 mm.

### Occlusive Element

An occlusion element of the present invention may have a variety of shapes including spiral, football, box, etc. and is located at the distal end of a canalicular occlusion device. An occlusion element is held in position by the memory frame that is connected to the occlusion element and is responsible for converting the occlusion element from a constrained form to an expanded form. An occlusion membrane may be attached to the frame according to techniques known by those skilled in the art such as with an adhesive. The adhesive may be a thermoplastic adhesive and more preferably may be a thermoplastic fluoropolymer adhesive such as fluorinated ethylene propylene (hereinafter FEP) or perfluoroalkoxy (hereinafter PFA). In some embodiments, the occlusive element may comprise first and second tubular coverings. When such an occlusive element is expanded, the tubular coverings are affixed to each other through the multiplicity of openings in the stent wall. The two coverings may be affixed by heating them above the crystalline melt point of a polymer they are made of, such as that of PTFE film, to adequately cause them to thermally adhere. Alternatively, an adhesive such as FEP may affix them. In addition to FEP, other thermoplastic polymers including thermoplastic fluoropolymers may also be used to make this coated film. The adhesive coating on the porous expanded PTFE film may be either continuous (non-porous) or discontinuous (porous) depending primarily on the amount and rate of stretching, the temperature during stretching, and the thickness of the adhesive prior to stretching. Occlusive elements are made of occlusive element materials. Suitable occlusive element materials including polytetrafluoroethylene, polyethylene terephthalate, polyethylene, silicone, acrylate polymer, urethane polymer, rayon, rubber, latex, polyurethane, thermoplastic polyurethane, polyvinylchloride, and a combinations of the above, as examples, and or coating of said materials on other natural or other synthetic fabrics in order to achieve a waterproof membrane

### Fixation Element

A fixation element is located preferably on the most proximally on a canalicular occlusion device and is designed to be delivered within punctal os in order to prevent migration and enable retrieval. The shape of said arms can be modified to a variety of shapes in order to improve ease of recovery, comfortability, and stability. A fixation element may be a wire coated with a layer of PTFE. Fixation element designed strategies include T arms, S arms, loops, helix, coil, Y shape, golf tee, and sphere shapes, as example. A symmetrical fixation element design such as a coil or S may be preferred as compared to a T design to enable precise alignment along an eyelid margin of a subject. For example, a circular shape fixation element can be deployed in any orientation 360 in relation to punctal os whereas a fixation element have arms in the shape of a wherein the arms are oriented towards and away from eye may prove uncomfortable and potentially harmful to eye if extension is beyond the lid margin. In order to prevent inward migration, as well as to enable ease of removal, a fixation element emerges from the punctum and expand outward during delivery, while the a memory frame and/or occlusion membrane convert from a constrained form to an expanded form in the canaliculus since the shape of the memory frame and or occlusion frame in its expanded form is larger than the punctal os. In the event removal of a canalicular occlusive device is necessary, upward traction is applied to the fixation arms. This traction will be transferred from the fixation arms to the remaining elements of canalicular occlusive device. The memory frame and/or occlusive element will collapse to converting to a more linear configuration (or constrained form) when loaded into the delivery device. This self-collapsing mechanism helps minimize canalicular trauma and patient discomfort when the device is removed. Such a design moreover enables the end user to remove the device with ease at the slit lamp with microforceps, as an example.

Manufacturing of the memory frame and linear frame are easily automated. Strategies for automation include a method to facilitate rapid and reproducible coiling of the helix consisting of a mask with grooves (such as a microdrill bit) that can be mounted onto a device that allows rotation of the frame but otherwise holds it in one position in the x, y and z axis. In a simple embodiment, the device consists of a microdrill handle and bit affixed to bearings that are then held stationary by a vice. The shape memory metal is spooled within the grooves of the device and then an external clamp such as a hemostat is applied to hold the wire wound around the mask. With clamp in place the memory metal is then heat treated in order to reset the shape of the memory metal in a coiled configuration.

A memory frame of the present invention may be made of memory metal. A memory frame may be partially or fully covered by an occlusive element made of a biocompatible membrane, as an example. All components of a canalicular occlusion device of the present invention may be made of the same or different materials.

### Canalicular Occlusion Device Delivery to a Subject

The canalicular occlusion device of this invention is designed to be held in a collapsed form when associated with the delivery device. In its deployed form, the memory frame is free to expand and thereby expands the attached occlusive element. In its expanded form the occlusive membrane is held approximate to the canalicular wall and substantially prevents fluid passage through the canalicular system. Prior to delivery, an occlusive membrane is held in an approximately linear form or constrained form. When a wire is inserted coaxially in the proximal portion of the cannula it pushes an occlusive device out of the lumen and thereby causes the memory frame to expand forming and creating an expanded form of an occlusive device. The expansion of the memory frame thereby causes expansion of an occlusive membrane into an expanded form. An occlusive element in it expanded form remains in the distal cannula after delivery.

The canalicular occlusion devices of the present invention are designed to adapt to all anatomy. Some subjects have very a short vertical portion of their canalicular system while other subjects have a canalicular system with very long vertical portions. Similar variations in anatomy can be found in the horizontal component of the canalicular system. A delivery device is able to provide an occlusion device having an occlusion element in a constrained form (i.e. stretched to a substantially linear form). Upon and/or during delivery the occlusive element of the present invention is able to gently expand into the canalicular system regardless of the anatomical variation as shown in FIG. 3 (i.e. expanded form of an occlusion device of the present invention). In addition, by choosing the appropriate final diameter of an expanded occlusion element on the distal portion of an occlusion device, the occlusive element will comfortably fasten the canalicular occlusion device within the canalicular system.

Canalicular Occlusion Device Removal from a Subject

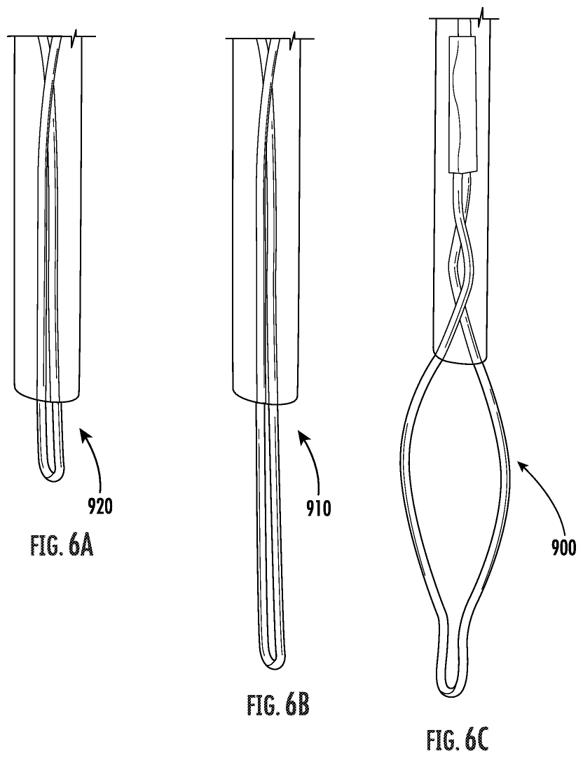

A canalicular occlusion device without fixation arms or linear frame is deployed within the canalicular system and maybe retrieved from a subject using a removal device that is inserted into the canalicular system and attaches to the canalicular device. A canalicular occlusion device having a fixation element and/or a linear frame maybe retrieved from a subject by a device that attaches to the fixation element. Suitable removal devices used in the present invention include micro forceps, nitinol loops or any other device that in some embodiments is inserted into the canalicular system, attaches to a canalicular occlusion device, and removes it from a subject. Please see FIG. 6. In an ideal design, the removal device should be low profile and of the right flexibility to easily thread into the canalicular os, abut a canalicular occlusion device of the present invention, engage (or attach to) the device. Tension is applied to the removal device so as to remove a canalicular occlusion device from the canalicular system of a subject.

An inclusion device of the present invention may be designed to enhance recovery. For example, fixation elements may be made in the shape of one or more hoops, or a helix to engage a removal device. The memory frame element may have a portion in which no sheet of PTFE connects the adjoining loops of nitinol coil. The memory frame may be a helix and the diameter of said neighboring loops can be varied in order to enable engagement of the helical frame by the removal device.

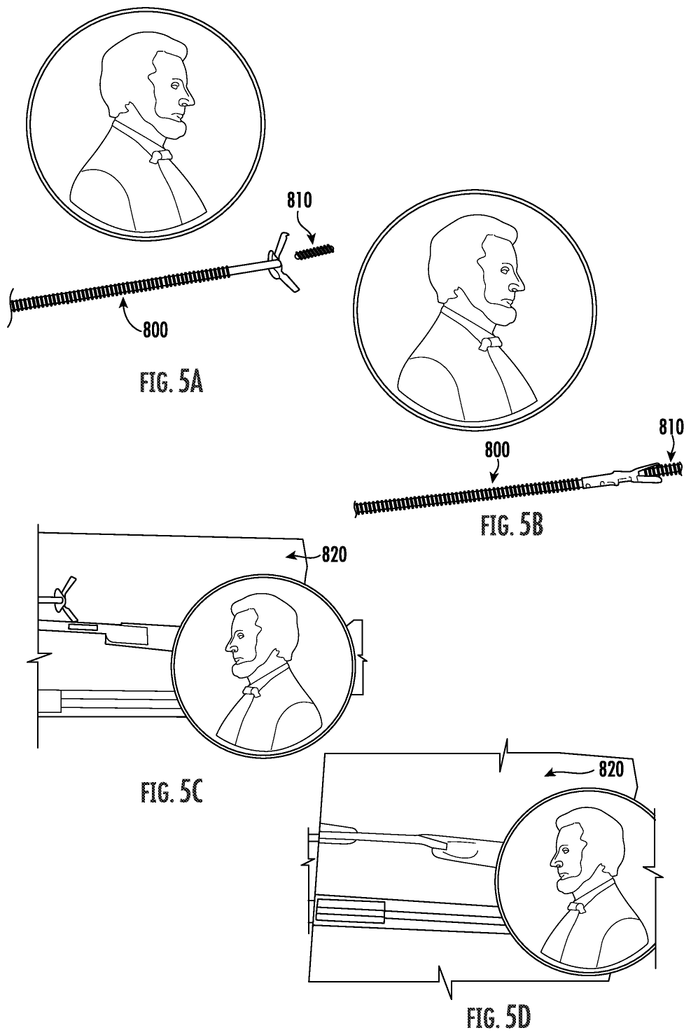

Additional examples of removal devices are described in FIG. 5. A microforcep 800 is able to attach to a prototype occlusion device 810 in a model canalicular system 820 as shown in FIG. 5C. The removal device will pull on an occlusive device of the present invention an remove it from the canalicular system. The removal device, such as microforceps 800, may attached to any location of an occlusive device for retrieval of a canalicular occlusive memory device from the canalicular system. For example, a removal device may attach to a fixation element in the shape of arms 408 as shown in FIG. 4. Other times a removal device may attach to an occlusive device of the present invention at other locations such as at the memory frame area, and/or the occlusive element area as examples. Applying pressure to the removal device while it is attached to an occlusive device of the present invention causes an occlusive device of the present invention to straighten out and take a constrained form while in the canalicular system during removal. The removal of a stretched occlusive device (i.e. in a constrained form) from a subject results in minimal trauma or tissue damage during removal of the occlusive device. Other examples of removal devices includes those embodiments illustrated in FIG. 6A-C. A removal device in the shape of an open loop 900 is ready to attach an occlusion device of the present invention located in a canalicular system of a subject. A removal device in the shape of a closed loop 910 illustrates how a delivery device will enter a canalicular system of a subject. A removal device in a sheath 920 illustrates a removal device surrounded by a sheath prior to the removal of an ocular device.

### EXAMPLES

### Example 1: Occlusion

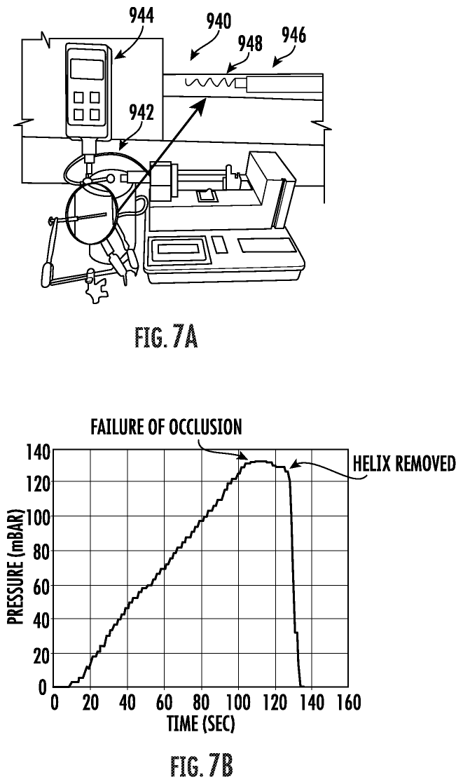

FIG. 7 illustrates an occlusion testing system consisting of an acrylic model 940 attached to a syringe pump elastomeric tubing 942 as well as a pressure gauge 944 thereby enabling measurement of the resulting pressure change in the acrylic model 940, more specifically the cylinder space 946 before and after occlusion of the cylinder space 946 with second prototype occlusion device 948. A red (or thick) arrow points to an illustration providing a view within an acrylic model 940 of a prototype second prototype occlusion device 948 being deployed. In this example, the second prototype occlusion device comprises heat shrink PTFE applied over a Cook commercial aneurysm coil. The helical structure of the second prototype occlusion device 948 is seen as it is pushed with non-floppy end of guidewire through deployment into the cylinder space 946. FIG. 7b illustrates a graph having pressure units on the Y axis versus time units on the X axis. Once a second prototype occlusion device 948 is deployed in the cylinder space 946, the pressure is increased. The pressure is allowed to build up behind the second prototype occlusion device 948 until the second prototype occlusion device fails to occlude in the cylinder space 946 allowing air to escape around the second prototype occlusion device 948. In addition, when a second prototype occlusion device 948 is removed from the cylinder space 946 using microfoceps, the pressure falls to zero within the cylinder space 946. The testing system has demonstrated that, the second prototype occlusion device 948 fails to occlude the cylinder space 946 when the pressure behind the second prototype occlusion device is in the range of 121 to 141 mbar. The helix was capable of withstanding 131.25+/−10.37 mbar (n=47) before allowing air to escape around the device (i.e. when graph reaches peak pressure as shown in FIG. 7b). Following occlusion failure, the second prototype occlusion device 948 is removed showing a rapid return to zero pressure illustrating complete reversible occlusion. With the inherent limitations of an acrylic model 940 being made of relatively solid material, further in vivo work is required to determine the optimal outward radial force of the an inclusion device of the present invention. Such force may be modified by changing: the number of coils, gauge of the wire, diameter of the coil and the materials used to make an occlusive device of the present invention.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A canalicular occlusion device comprising:
a memory frame comprising a memory material; and
an occlusive element configured to substantially prevent fluid from flowing through portions of the canaliculus wherein the occlusive element is attached to the memory frame.

2. The canalicular occlusion device of claim 1 wherein the memory frame is a length equal to or shorter than the distance from a subject's ocular punctum to the end of the subject's canalicular space.

3. The canalicular occlusion device of claim 1 further comprising a linear frame.

4. The canalicular occlusion device of claim 1 wherein the memory material is selected from the group consisting
of polynorbornene, polycaprolactone, polyenes, nylons, polycyclooctene (PCO), blends of PCO and styrene-butadiene rubber,
polyvinylacetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, and combinations thereof.

5. The canalicular occlusion device of claim 1 wherein the memory material is a metal selected from the group consisting of stainless steel, cobalt, nickel, chromium, molybdenum, titanium, nitinol, tantalum, platinum-iridium alloy, gold, magnesium, and a combination thereof.

6. The canalicular occlusion device of claim 1 wherein at least a part of the memory frame is completely covered by the occlusive element.

7. The canalicular occlusion device of claim 1 wherein the occlusive element comprises a memory material selected from the group consisting of polytetrafluoroethylene, polyethylene terephthalate, polyethylene, silicone, acrylate polymer, urethane polymer, rayon, rubber, latex, polyurethane, thermoplastic polyurethane, polyvinylchloride and a combination thereof.

8. The canalicular occlusion device of claim 3 further comprising a fixation element, wherein the linear frame is located between the occlusive element and the fixation element.

9. The canalicular occlusion device of claim 8 wherein the memory frame, the fixation element, and the occlusive element comprise the same material.

10. The canalicular occlusion device of claim 8 wherein the memory frame, the fixation element, and the occlusive element comprise different materials.

11. The canalicular occlusion device of claim 8 wherein the memory frame comprises nitinol and the fixation element comprises a polymer.

12. The canalicular occlusion device of claim 1 wherein the memory frame has a rigidity in a range of 1 kPa to 10 kPa.

13. The canalicular occlusion device of claim 1 having a length in a range of 0.1 mm to 10 mm.

14. The canalicular occlusion device of claim 1 having a diameter in a range of 0.1 mm to 10 mm.

15. A method of occluding a canalicular system, the method comprising: delivering a canalicular occlusion device of claim 1 configured in a constrained form associated with a delivery device to a target location within a canalicular system, wherein when the canalicular occlusion device is deployed the canalicular device thereby assumes an expanded form making contact with walls of the canaliculus.

16. The method of claim 16 wherein the constrained form does not prevent fluid from flowing through the canaliculus.

17. The method of claim 16 wherein the expanded form prevents fluid from flowing through the canaliculus.

18. The canalicular occlusion device of claim 1 wherein the occlusive element comprises a waterproof coating.

19. A canalicular occlusion device comprising:
a memory frame comprising a memory material, a fixation element, and a linear frame located between the memory frame and the fixation element.

20. The canalicular occlusion device of claim 19 further comprising an occlusive element configured to substantially prevent fluid from flowing through portions of the canaliculus wherein the occlusive element is attached to the memory frame
